(19) United States
(12) Patent Application Publication
(10) Pub. No.: US 2025/0115715 A1
(43) Pub. Date: Apr. 10, 2025

(54) POLYMER COMPOUNDS AND COMPOSITION FOR NUCLEIC ACID DELIVERY COMPRISING THE SAME

(71) Applicant: Inha University Research and Business Foundation, Incheon (KR)

(72) Inventors: Hyun Jin KIM, Incheon (KR); Sungbeen LIM, Incheon (KR); Dong Hyun KANG, Incheon (KR)

(73) Assignee: Inha University Research and Business Foundation, Incheon (KR)

(21) Appl. No.: 18/906,527

(22) Filed: Oct. 4, 2024

(30) Foreign Application Priority Data
Oct. 6, 2023 (KR) 10-2023-0133523
Sep. 25, 2024 (KR) 10-2024-0129490

(51) Int. Cl. C08G 73/02; A61K 9/16; A61K 31/787; B82Y 30/00

(52) U.S. Cl. CPC C08G 73/024; A61K 9/1641; A61K 9/1682; A61K 31/787; B82Y 30/00

(57) ABSTRACT

Provided are a polymer compound and a composition for nucleic acid delivery comprising the same, wherein the polymer compound has a high binding affinity to nucleic acids and effectively protects nucleic acids from nucleases, thereby improving the nucleic acid delivery effect and stability.

### BACKGROUND

### Technical Field

The present invention relates to an amphiphilic polymer compound and a composition for nucleic acids delivery comprising the same.

### Background Art

mRNA vaccines, which are attracting attention as next-generation vaccines, artificially synthesize and inject mRNA containing the virus' genetic information to activate immunity by causing cells to directly produce spike proteins. Since mRNA vaccines utilize the genes of the virus, they can be developed quickly once the genetic structure of the virus is identified, and there is no need to cultivate antigens or antibodies externally, so they have the advantage of being produced quickly. mRNA vaccines have been used worldwide to respond to the COVID-19 pandemic, and various research and development are currently being conducted using them to overcome various diseases, such as cancer and rare diseases, as well as infectious diseases.

Meanwhile, since mRNA can be easily damaged by enzymes in the human body, nano-delivery vehicles are attracting attention as a technology for safely delivering mRNA to cells. The most widely used nano-delivery vehicle currently is the lipid nano particle (LNP), and the recent COVID-19 vaccines developed by Pfizer and Moderna also use LNP delivery vehicles. However, in the case of LNPs, lipids are easily oxidized, making storage and distribution at room temperature difficult, and side effects such as hypersensitivity reactions caused by the toxicity of LNPs themselves have been reported.

To overcome this, research on synthetic polymer nano-delivery vehicles is being actively conducted. Synthetic polymer nano-delivery vehicles are made by repeatedly combining small units, and their structure, size, ionization characteristics, and stimulus-responsive drug release can be freely designed, and they can also be used in gene therapy that allows gene editing in the future. In addition, by introducing other components into the polymer nano-delivery vehicle, more advantages can be realized simultaneously while maintaining the properties of the polymer, and the composition can be controlled by multi-polymer systems and polymer-based hybrid systems, such as polymer-lipid and polymer-inorganic hybrid delivery nanoplatforms.

mRNA-loaded polymer nanoparticles have various structures, including polyplexes, polymer micelles, polymersomes, and core-shell structured polymer-lipid hybrids. These polymer nanoparticle delivery vehicles have low toxicity, excellent biodegradability and biocompatibility, which can reduce toxicity and immune responses, and can provide basic data for the development of next-generation vaccines and pharmaceuticals where mRNA delivery with LNPs is difficult. In addition, polymer-based mRNA delivery technology can be used not only for the development of mRNA vaccines but also for the development of other RNA drugs, and thus has high potential for use in the treatment of various diseases.

As an example of polymeric nanoparticle delivery vehicles, cationic dendrimers have been used in nonclinical trials against a variety of viruses, including Ebola, H1 N1 influenza, and Zika, however, due to the structural characteristics of the multi-branched molecules themselves, it is difficult to decompose in vivo, resulting in toxicity, and thus requires improvement. Polymer nanoparticles have been reported in numerous literatures to be used alone or in combination with lipids as lipid-polymer complexes; however, compared to the research results, it is difficult to accurately analyze the physical properties and elucidate the structure, so the clinical entry barrier is somewhat high, and thus they have not yet been commercialized.

### DETAILED DESCRIPTION OF THE INVENTION

### Summary

The present invention has been made to solve the above problems, and aims to provide a high-performance polymer delivery vehicle and a composition for nucleic acids delivery comprising the same.

### Technical Solution

The present invention provides a compound represented by Formula 1.

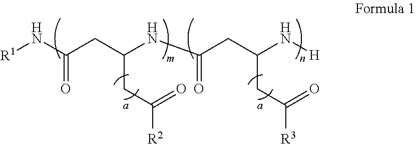

Wherein,
R¹ is an C₁˜C₁₀ alkyl group; —(CH₂)₃—O—CH₃; or —(CH₂)ₓ—[(CH₂)₂—O]_{y}—CH₃;
x is 0 or 1, y is an integer from 1 to 45,
provided that when y is 1, x is 1, and if y is 2 or more, x is 0,
m and n are independently integers from 1 to 100,
provided that m+n≥10,
a is an integer from 0 to 2, and
R² and R³ are each independently represented by Formula 2,

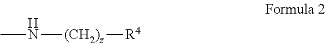

Wherein,
R⁴ is selected from the group consisting of hydrogen; an amino group; a hydroxy group; a C₁˜C₂₀ alkoxy group; a C₃˜C₂₀ aliphatic ring group; a C₆˜C₂₀ aryl group; a C₁˜C₂₀ heteroaryl group; a C₁˜C₂₀ heterocyclic group; —NH—C₁˜C₂₀ alkenyl group-NH₂; —NH—C₁˜C₂₀ hydroxyalkyl group; —N(C₁˜C₂₀ alkyl group)(C₁˜C₂₀ alkyl group); —N(C₁˜C₂₀ alkyl group)(C₁˜C₂₀ alkenyl group-NH₂); —(OH)(C₁˜C₂₀ alkyl group); and —(C₁˜C₂₀ hydroxyalkyl group)(C₁˜C₂₀ hydroxyalkyl group);
z is an integer from 0 to 45,

The R¹ can be represented by any one of Formulas 1-1 to 1-3.

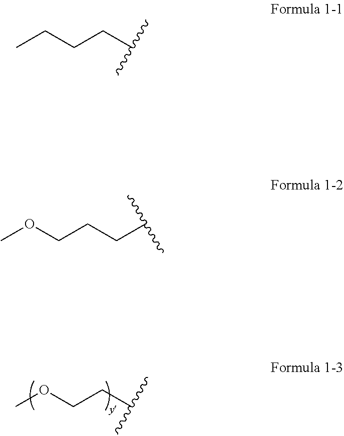

Wherein:
y′ is an integer from 2 to 45,
[symbol] indicates the position to be bonded.

The R² or R³ is represented by any one of Formulas 2-1 to 2-20.

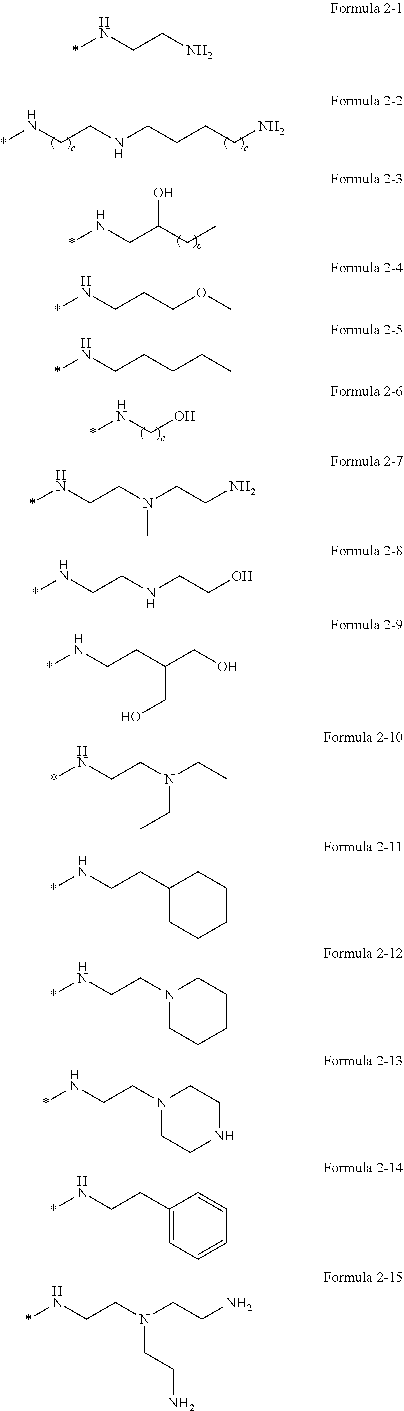

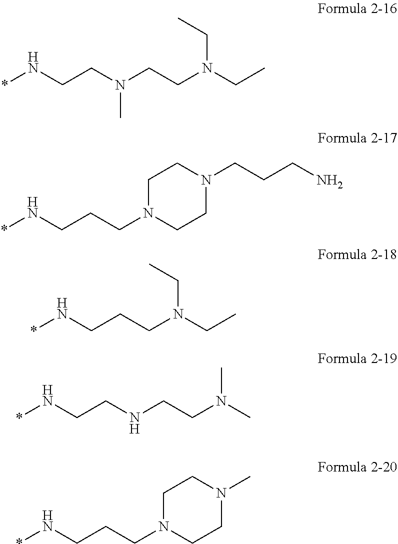

Wherein:
c is an integer from 0 to 12.

In another aspect, the present invention provides a composition for nucleic acids delivery comprising the compound.

The nucleic acids may be selected from the group consisting of DNA, RNA, PNA, interfering RNA (siRNA), asymmetric interfering RNA (aiRNA), microRNA (miRNA), dicer-substrate RNA (dsRNA), small hairpin RNA (shRNA), messenger RNA (mRNA), transfer RNA (tRNA), ribosomal RNA (rRNA), antisense oligonucleotides and mixtures thereof.

Additionally, the composition for nucleic acid delivery may further comprise a pharmaceutically acceptable carrier or a pharmaceutically acceptable salt.

Also, in another aspect, the present invention provides a nanoparticle formed by bonding the compound and a nucleic acid.

Molar ratio of amino groups in polymer to phosphate group in nucleic acid (N/P ratio) is 1:1 to 30:1.

Also, in another aspect, the present invention provides a vaccine composition comprising the compound.

In addition, in another aspect, the present invention provides a method for producing a compound represented by Formula 1, comprising:
1) A step of synthesizing a beta-amino acid N-thiocarboxyanhydrides (beta-NTAs) monomer;
2) A step of synthesizing a polymer precursor by ring-opening polymerization of the beta-NTAs monomer using butylamine as an initiator; and
3) A step of modifying the side chain of the polymer precursor through an aminolysis reaction;

In another aspect, the present invention provides a method for delivering a nucleic acid into a cell using the compound.

The intracellular location is the cytoplasm or nucleus.

### Effects of the Invention

The amphiphilic polymer compound according to the present invention can be easily manufactured through a simple reaction, exhibits high binding capacity to therapeutic nucleic acids such as RNA drugs, and effectively protects nucleic acids from nucleases, thereby improving stability and delivery ability in blood as a nucleic acid delivery vehicle.

### BRIEF DESCRIPTION OF THE DRAWINGS

FIG. 1 is a ¹H NMR spectrum of a Benzyl-β³-LCHG monomer according to one embodiment of the present invention.

FIG. 2 is a ¹H NMR spectrum of a Benzyl-β³-LCHA monomer according to one embodiment of the present invention.

FIG. 3 is a ¹H NMR spectrum of a monomer (a=2) according to one embodiment of the present invention.

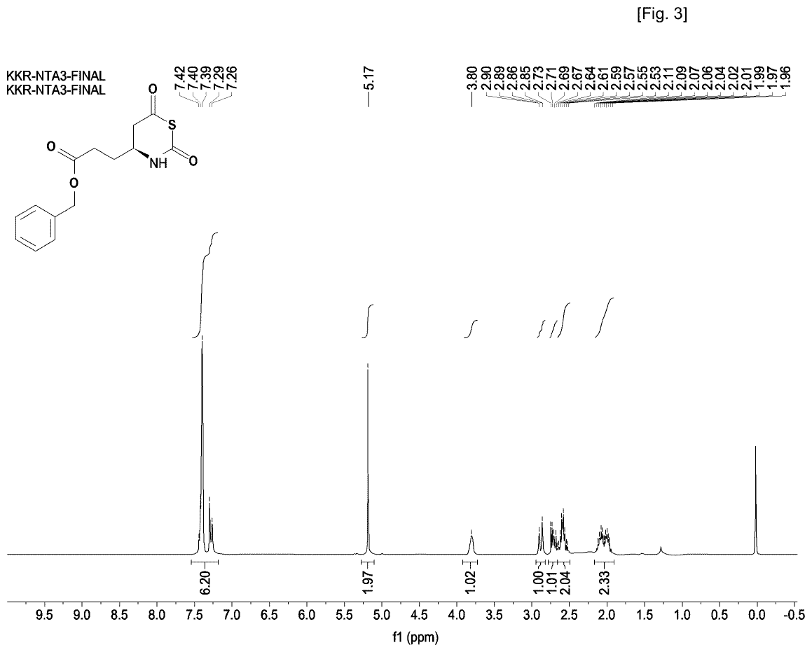

FIG. 4 is an (A) SEC chart and (B) ¹H NMR spectrum (5 mg/mL, DMSO-d₆, 400 MHz, rt) of a butyl-poly(benzyl-β³-LCHG) precursor polymer according to one embodiment of the present invention.

FIG. 5 is an (A) SEC chart and (B) ¹H NMR spectrum (5 mg/mL, DMSO-d₆, 400 MHz, rt) of a PEG₁-poly(benzyl-β³-LCHG) precursor polymer according to one embodiment of the present invention.

FIG. 6 is an (A) SEC chart and (B) ¹H NMR spectrum (5 mg/mL, DMSO-d₆, 400 MHz, rt) of a PEG₄-poly(benzyl-β³-LCHG) precursor polymer according to one embodiment of the present invention.

FIG. 7 is an (A) SEC chart and (B) ¹H NMR spectrum (5 mg/mL, DMSO-d₆, 400 MHz, rt) of a PEG₁₂-poly(benzyl-β³-LCHG) precursor polymer according to one embodiment of the present invention.

FIG. 8 is an (A) SEC chart and (B) ¹H NMR spectrum (5 mg/mL, DMSO-d₆, 400 MHz, rt) of a PEG₂₄-poly(benzyl-β³-LCHG) precursor polymer according to one embodiment of the present invention.

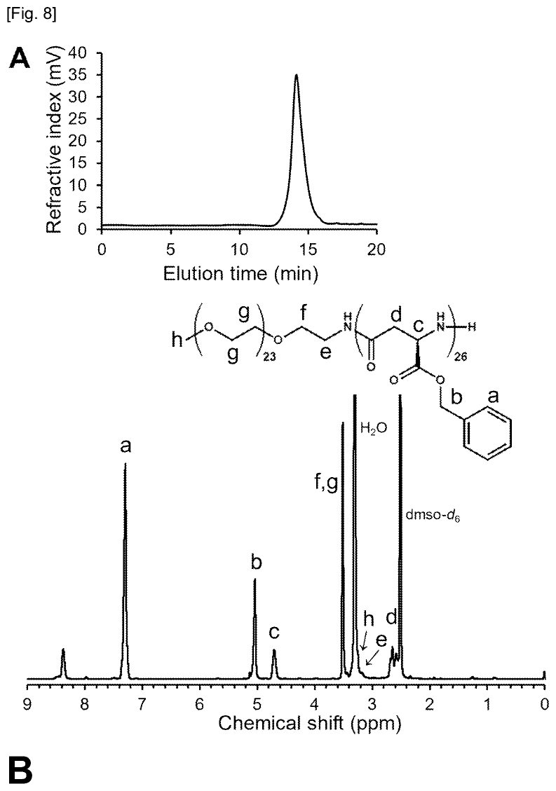

FIG. 9 is an (A) SEC chart and (B) ¹H NMR spectrum (5 mg/mL, DMSO-d₆, 400 MHz, rt) of a butyl-poly(benzyl-β³-LCHG) precursor polymer according to one embodiment of the present invention.

FIG. 10 is a ¹H NMR spectrum (5 mg/mL, D₂O, 400 MHz, rt) of a butyl-PGly(DET/CHE) derivative according to one embodiment of the present invention.

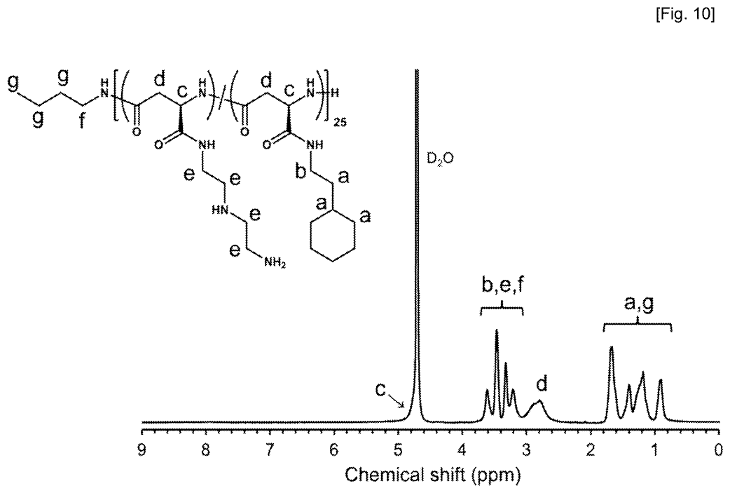

FIG. 11 is a ¹H NMR spectrum (5 mg/mL, D₂O, 400 MHz, rt) of a PEG₁-PGly(DET/CHE) derivative according to one embodiment of the present invention.

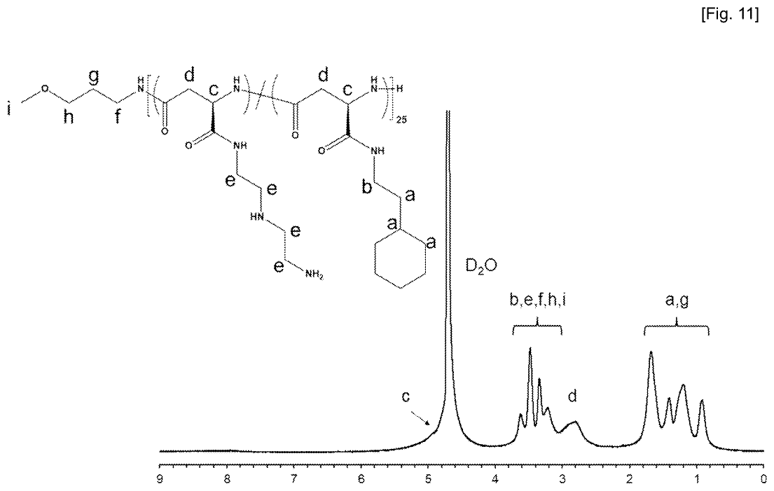

FIG. 12 is a ¹H NMR spectrum (5 mg/mL, D₂O, 400 MHz, rt) of a PEG₄-PGly(DET/CHE) derivative according to one embodiment of the present invention.

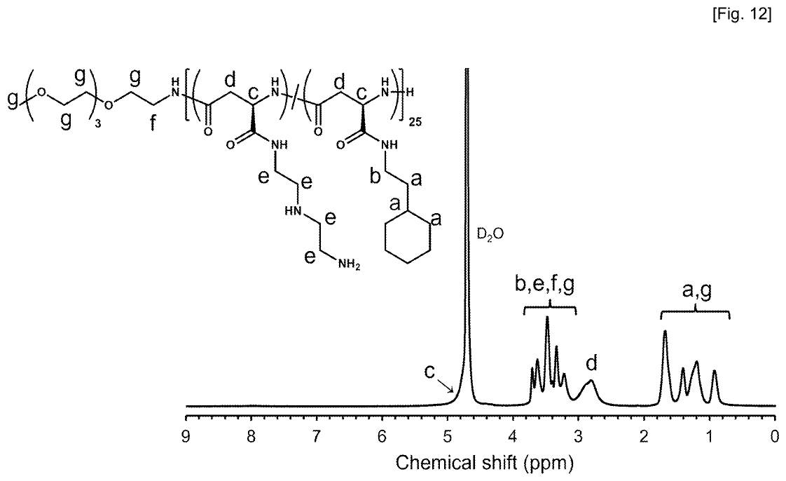

FIG. 13 is a ¹H NMR spectrum (5 mg/mL, D₂O, 400 MHz, rt) of a PEG₁₂-PGly(DET/CHE) derivative according to one embodiment of the present invention.

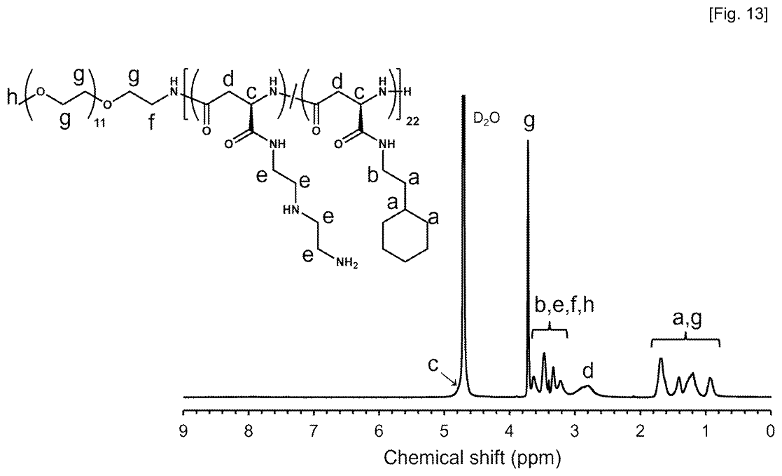

FIG. 14 is a ¹H NMR spectrum (5 mg/mL, D₂O, 400 MHz, rt) of a PEG₂₄-PGly(DET/CHE) derivative according to one embodiment of the present invention.

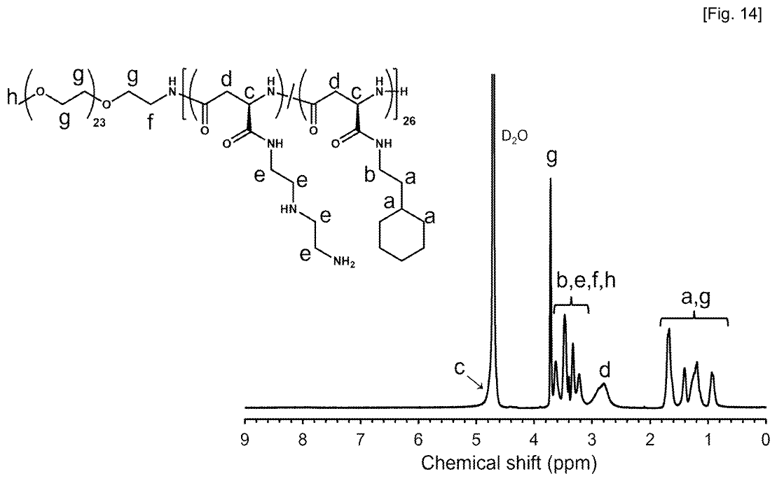

FIG. 15 is a ¹H NMR spectrum (5 mg/mL, D₂O, 400 MHz, rt) of a Butyl-PGly(DET/ADO) derivative according to one embodiment of the present invention.

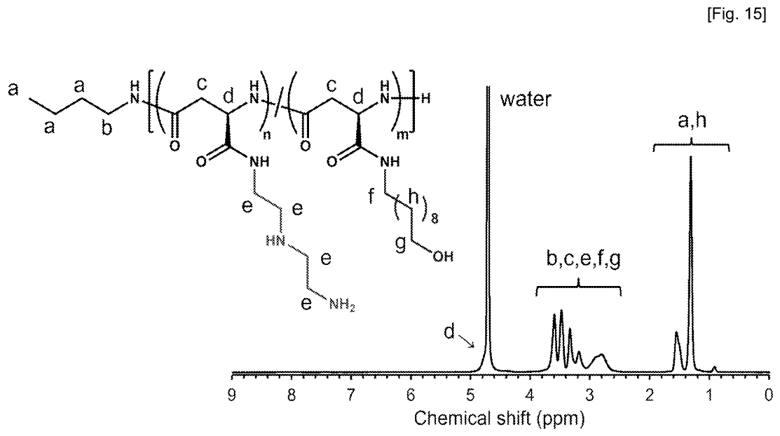

FIG. 16 is a ¹H NMR spectrum (5 mg/mL, D₂O, 400 MHz, rt) of a PEG₁-PGly(DET/ADO) derivative according to one embodiment of the present invention.

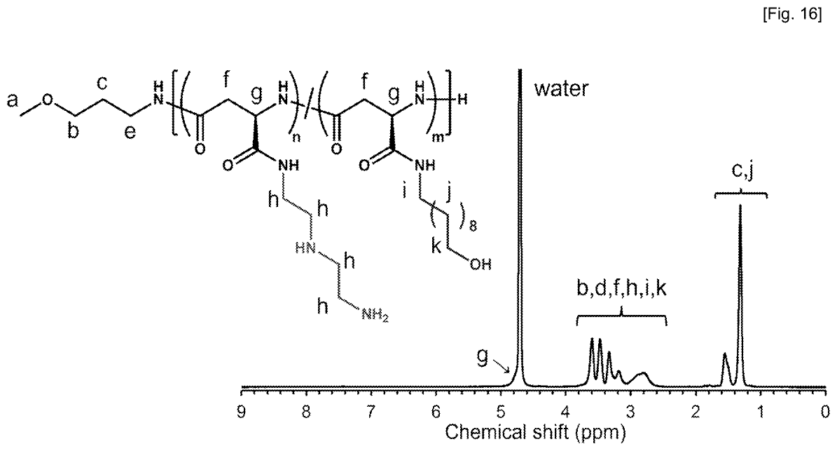

FIG. 17 is a ¹H NMR spectrum (5 mg/mL, D₂O, 400 MHz, rt) of a PEG₄-PGly(DET/ADO) derivative according to one embodiment of the present invention.

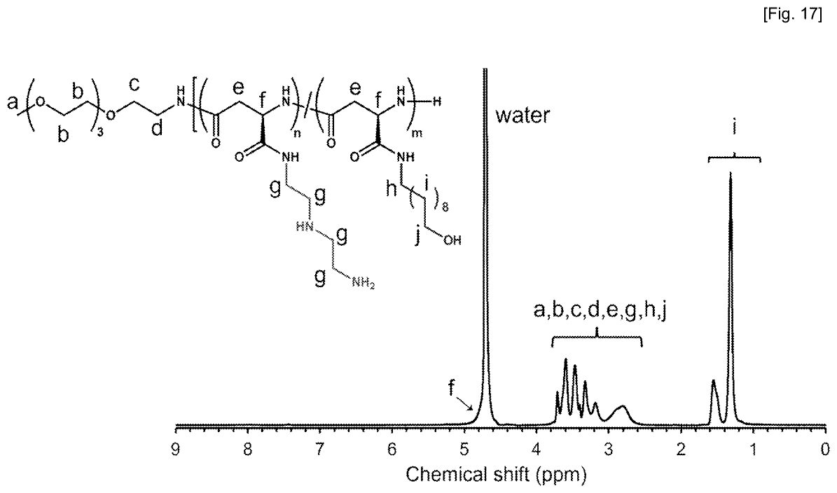

FIG. 18 is a ¹H NMR spectrum (5 mg/mL, D₂O, 400 MHz, rt) of a Butyl-PGly(AEP/ADO) derivative according to one embodiment of the present invention.

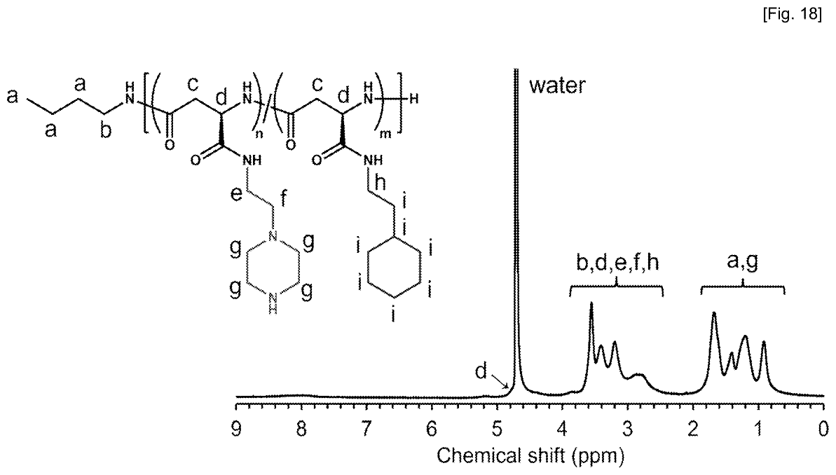

FIG. 19 is a ¹H NMR spectrum (5 mg/mL, D₂O, 400 MHz, rt) of a Butyl-PGly(HED/ADO) derivative according to one embodiment of the present invention.

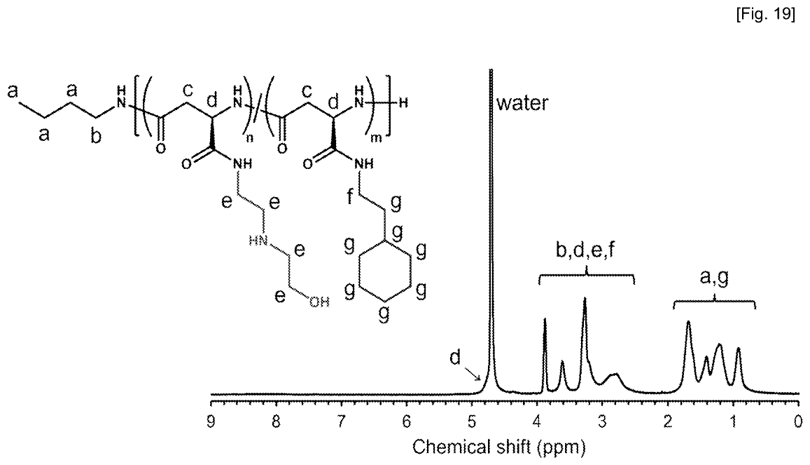

FIG. 20 is a ¹H NMR spectrum (5 mg/mL, D₂O, 400 MHz, rt) of a Butyl-PGly(MDE/ADO) derivative according to one embodiment of the present invention.

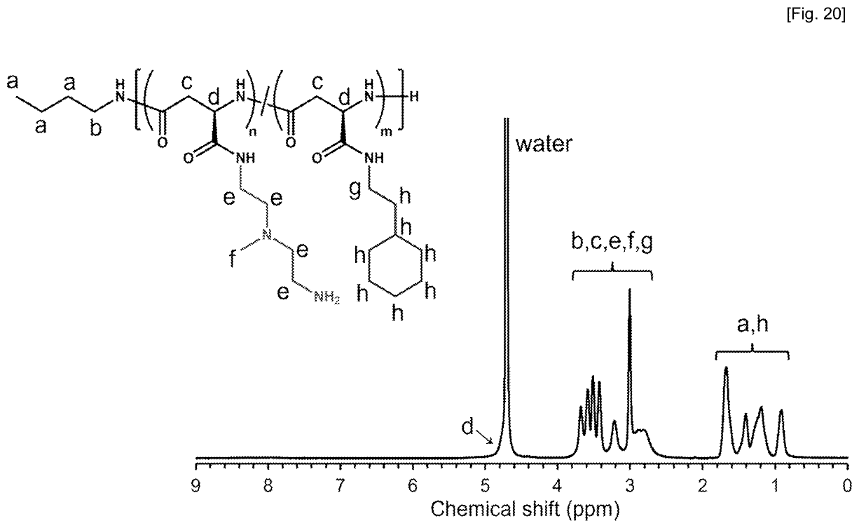

FIG. 21 is the results of measuring the size and surface charge of polymer nanoparticles of poly(benzyl-b³-LCHG) derivatives and mRNA according to one embodiment of the present invention.

FIG. 22 shows TEM images (A-C) and size distribution histograms (E-G) of polymer nanoparticles. (A,E)butyl-PGly(DET/CHE), (B,F)PEG₄-PGly(DET/CHE), and (C,G)PEG₂₄-PGly(DET/CHE).

FIG. 23 shows the results of measuring the expression level of luciferase mRNA of polymer nanoparticles in C2C12 cells according to one embodiment of the present invention, showing (A) a state where FBS was added and (B) a state where FBS was not added.

FIG. 24A shows the intracellular uptake efficiency of polymer nanoparticles in C2C12 cells, and FIG. 24B shows the results of stability analysis of polymer nanoparticles.

FIG. 25 shows the results of cell viability analysis for PEGₙ-PGly(DET/CHE) polymer nanoparticles in C2C12 cells.

FIG. 26A shows a schematic diagram of the fluorescence expression design of HEK293-loxP-GFP-RFP cells, and FIG. 26B shows the results of analyzing the amount of fluorescence expression in cells treated with polymer nanoparticles using Image J software.

FIG. 27 shows CLSM images observed 48 hours after transfection of various samples containing Cre mRNA into HEK293-loxP-GFP-RFP cells.

### DETAILED DESCRIPTION

Hereinafter, the present invention will be described in more detail.

The term “amphiphilicity” in this specification refers to the property of having both a water-soluble region and a hydrophobic region within a single molecular structure.

In the present invention, the term “alkyl” or “alkyl group” means an aliphatic hydrocarbon radical, and refers to a radical of a saturated aliphatic functional group comprising a straight-chain alkyl group, a branched-chain alkyl group, a cycloalkyl (alicyclic) group, an alkyl-substituted cycloalkyl group, and a cycloalkyl-substituted alkyl group. For example, C₁˜C₆ alkyl is an aliphatic hydrocarbon having 1 to 6 carbon atoms, and comprises methyl, ethyl, propyl, n-butyl, n-pentyl, n-hexyl, isopropyl, isobutyl, sec-butyl, tert-butyl, neopentyl, isopentyl, etc.

The term “alkenyl group” or “alkynyl group” as used in the present invention means a group in which at least 2 carbon atoms are formed by at least one carbon-carbon double bond or in which at least 2 carbon atoms are formed by at least one carbon-carbon triple bond, and comprises, but is not limited to, a straight-chain or branched-chain group.

The term “alkoxyl group” or “alkoxy group” used in the present invention, unless otherwise defined, means a radical in which a hydrogen atom of a hydroxy group is replaced with alkyl, and for example, an C₁˜C₆ alkoxy comprises methoxy, ethoxy, propoxy, n-butoxy, n-pentyloxy, isopropoxy, sec-butoxy, tert-butoxy, neopentyloxy, isopentyloxy, etc.

The term “heterocycle” or “heterocyclic group” as used in the present invention includes, unless otherwise stated, a compound comprising one or more heteroatoms or heteroatom groups such as SO₂, and comprises at least one of a single ring and a multiple ring, and comprises a heteroaliphatic ring and a heteroaromatic ring. It can also be formed by bonding adjacent functional groups.

The term “aryl group” or “arylene group” used in the present invention means a single ring or multiple ring aromatic group, and comprises an aromatic ring formed by the participation of adjacent substituents in a bond or reaction. For example, the aryl group can be a phenyl group, a biphenyl group, a fluorene group, or a spirofluorene group.

The term “aliphatic ring” used in the present invention means an aliphatic hydrocarbon ring.

The term “aromatic ring” used in the present invention means an aromatic system composed of a hydrocarbon comprising one or more rings, examples of which include benzene and naphthalene.

Additionally, the definitions described in the present invention can be added to form chemically related combinations, such as “arylalkyl”, “alkylcarbonyl”, “arylcarbonyl”, etc. When the term “alkyl” is used as a suffix, as in another term, such as “phenylalkyl” or “hydroxyalkyl”, it means an alkyl group substituted with a substituent selected from another specifically named group. Thus, for example, “Phenylalkyl” means an alkyl group having a phenyl substituent, and thus comprises benzyl, phenylethyl, and biphenyl. “Alkylaminoalkyl” means an alkyl group with an alkylamino substituent.

Essential factors for the production of high-performance polymer nanoparticles for nucleic acid delivery include the pKₐ of the polymer, hydrophobicity, the length and density of PEG on the nanoparticle surface, polymer degradability, and nanoparticle size.

The pKₐ of the polymer, i.e. the pKₐ value of the amino group, is responsible for binding or association with RNA and determines the endosomal escape ability and surface charge of the nanoparticle. When the pKₐ value of a polymer is 5-7 and enters a cell, it becomes highly charged within the endosome (pH 5.5), making it easy for the contained nucleic acid to escape from the endosome, resulting in excellent nucleic acid delivery efficiency in the body. Additionally, safety and nucleic acid delivery rate can be improved when the surface charge is in the high (+) or neutral range.

The hydrophobicity of the polymer can control the stability of the nanoparticles in hydrophilic solvents, and the surface charge of the nanoparticles can be controlled to be neutral by controlling the PEG length and density.

The present invention relates to a poly(beta-amino acids) converting agent, which is a cationic amphiphilic polymer capable of delivering RNA drugs including mRNA, small interfering RNA (siRNA), and antisense oligonucleotide (ASO) to cells, and a method for producing the same.

First, Benzyl-β³-LCHG monomer and Benzyl-β³-LCHA monomer were synthesized, next, the precursor polymers poly-(β³-benzyl-L-carboxyhomoglycine) [hereinafter, poly(benzyl-β³-LCHG)] and poly-(β³-benzyl-L-carboxyhomoalanine) [hereinafter, Butyl-poly(benzy-β³-LCHA)] were synthesized, and then poly(beta-amino acids) derivatives of various structures were prepared through secondary transformation with compounds having various pKₐ and hydrophobicity. Nanoparticles containing mRNA were manufactured using the synthesized polymer, and the mRNA delivery ability was evaluated through in vitro transfection.

The present invention provides a compound represented by Formula 1.

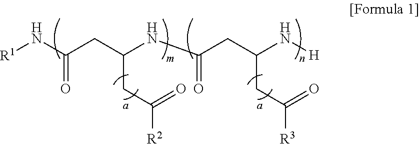

Wherein:
R¹ is an C₁˜C₁₀ alkyl group; —(CH₂)₃—O—CH₃; or —(CH₂)ₓ—[(CH₂)₂—O]_{y}—CH₃;
x is 0 or 1, y is an integer from 1 to 45,
provided that when y is 1, x is 1, and when y is 2 or greater, x is 0,
m and n are independently integers from 1 to 100,
provided that m+n≥10,
a is an integer from 0 to 2,
R² and R³ are independently represented by Formula 2,

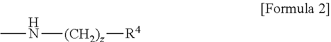

Wherein:
R⁴ is selected from the group consisting of hydrogen; amino group; hydroxy group; C₁˜C₂₀ alkoxy group; C₃˜C₂₀ aliphatic group; C₆˜C₂₀ aryl group; C₁˜C₂₀ heteroaryl group; C₁˜C₂₀ heterocyclic group; —NH—C₁˜C₂₀ alkenyl group-NH₂; —NH—C₁˜C₂₀ hydroxyalkyl group; —N(C₁˜C₂₀ alkyl group)(C₁˜C₂₀ alkyl group); —N(C₁˜C₂₀ alkyl group)(C₁˜C₂₀ alkenyl group-NH₂); —(OH)(C₁˜C₂₀ alkyl group); and —(C₁˜C₂₀ hydroxyalkyl group)(C₁˜C₂₀ hydroxyalkyl group);
z is an integer from 0 to 45.

Additionally, R¹ is represented by any one of Formulas 1-1 to 1-3.

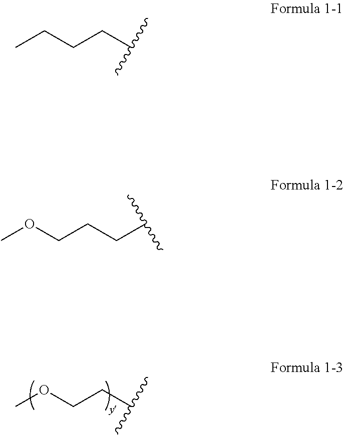

Wherein:
y′ is an integer from 2 to 45, preferably y′ is an integer from 2 to 25,
[symbol] indicates the position to be bonded.

Also, R² or R³ is represented by any one of Formulas 2-1 to 2-20.

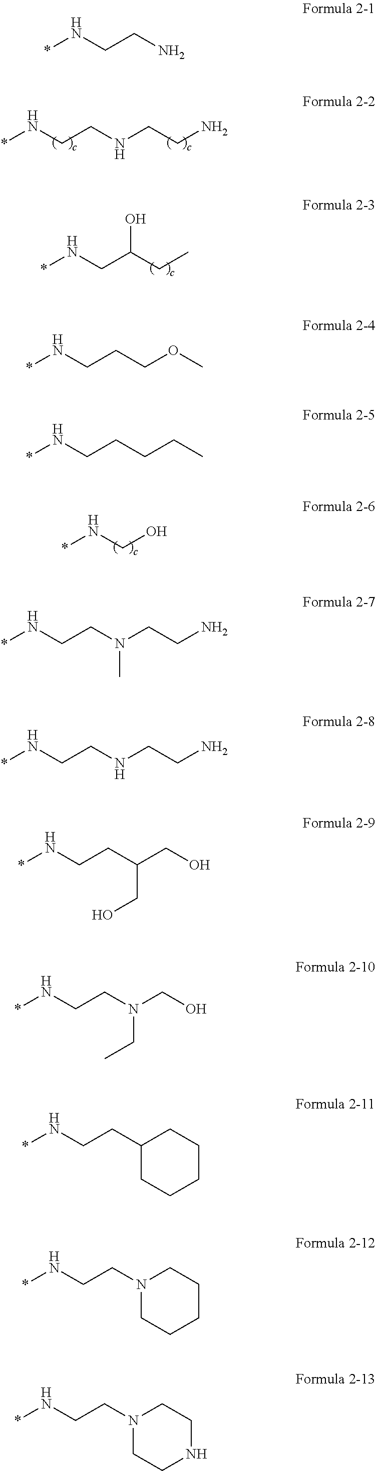

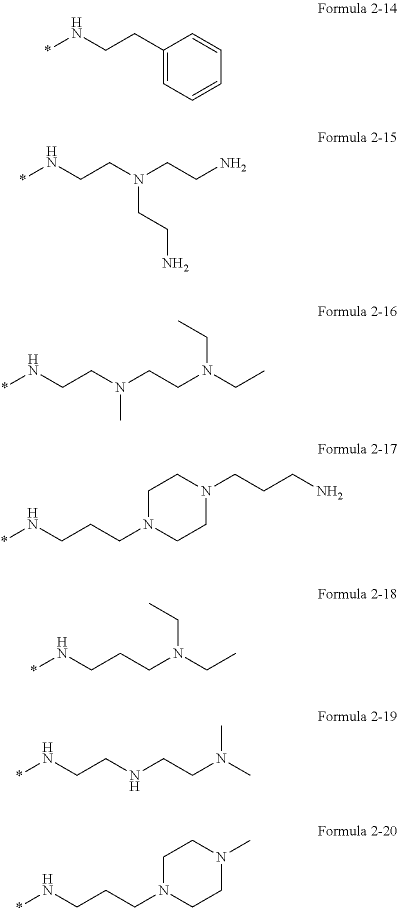

Wherein:
c is an integer from 0 to 12.

R² is a functional group for controlling the pKₐ of the polymer, and R³ is a functional group for controlling the hydrophobicity of the polymer.

Specifically, the compound represented by Formula 1 may be represented by any one of the following compounds, but is not limited thereto.

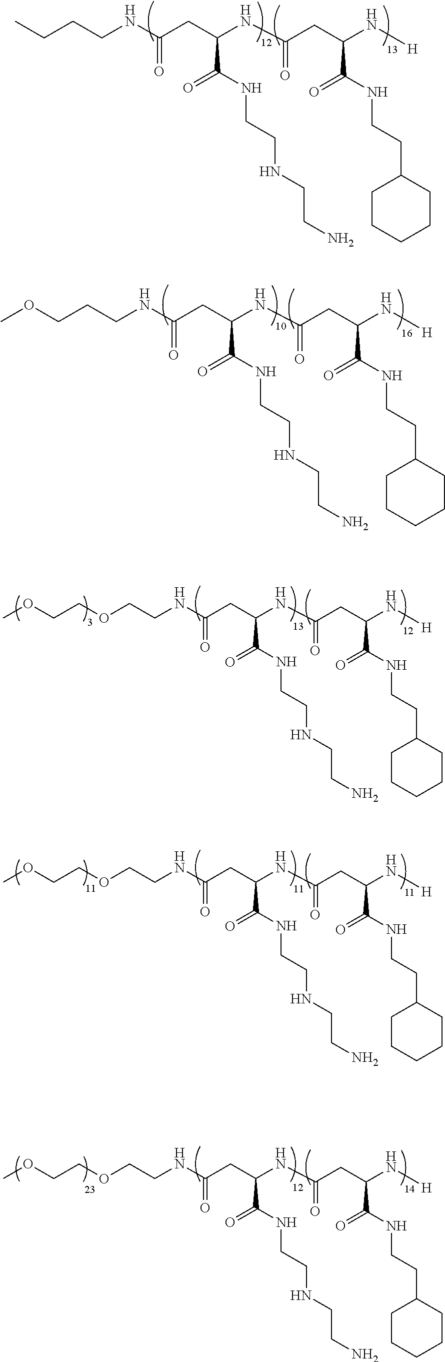

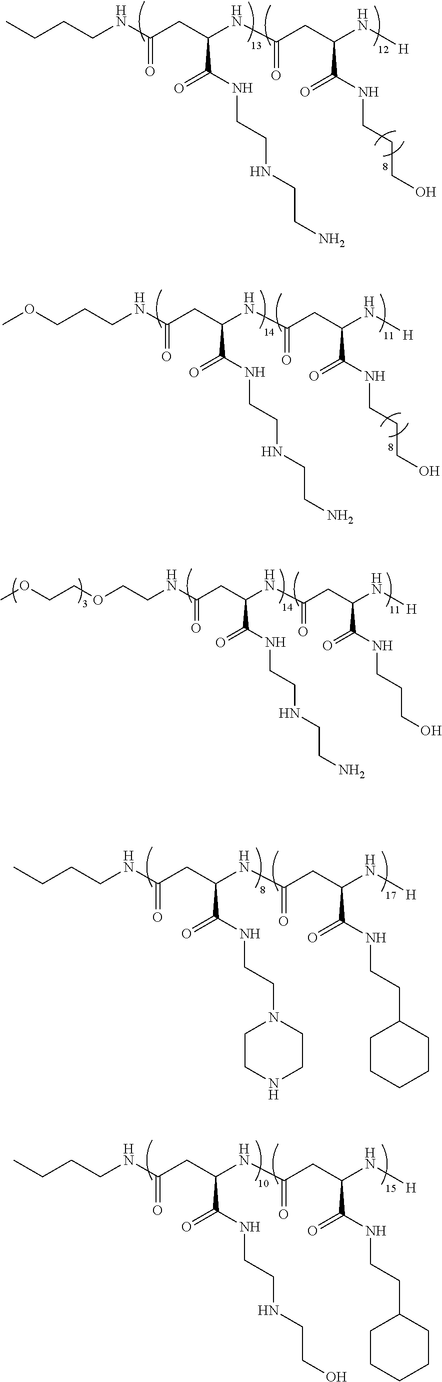

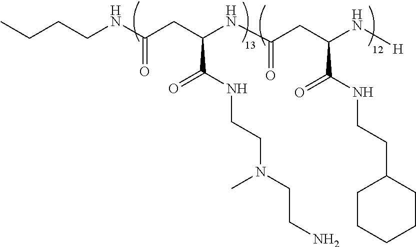

In addition, in another aspect, the present invention provides a composition for nucleic acids delivery comprising the compound.

The nucleic acid may be selected from the group consisting of DNA, RNA, PNA, interfering RNA (siRNA), asymmetric interfering RNA (aiRNA), microRNA (miRNA), dicer-substrate RNA (dsRNA), small hairpin RNA (shRNA), messenger RNA (mRNA), transfer RNA (tRNA), ribosomal RNA (rRNA), antisense oligonucleotides (ASOs) and mixtures thereof, but is not limited thereto, and preferably may be mRNA, siRNA or ASO.

The compound can bind to a nucleic acid to form a polymer nanoparticle. The compound is a cationic amphiphilic polymer that binds well to nucleic acids, so the binding is not broken even when the blood environment changes, etc., and the gene can be effectively delivered to the target site.

The composition for nucleic acids delivery may further comprise a pharmaceutically acceptable carrier or a pharmaceutically acceptable salt.

Also, in another aspect, the present invention provides a nanoparticle formed by bonding the compound and a nucleic acid.

In the nanoparticles, the molar ratio of protonable amino group in the compound (or polymer) to phosphate group in the nucleic acid (N/P ratio) may be 1 to 30, preferably 3 to 10, and more preferably 5 to 10.

Additionally, in another aspect, the present invention provides a vaccine composition comprising the compound.

Also, in another aspect, the present invention provides a method for producing a compound represented by Formula 1, comprising
1) a step of synthesizing a beta-amino acid N-thiocarboxyanhydrides (beta-NTAs) monomer;
2) a step of synthesizing a polymer precursor by ring-opening polymerization of the beta-NTAs monomer using butylamine as an initiator;
3) a step of modifying a side chain of the polymer precursor through an aminolysis reaction.

In another aspect, the present invention provides a method of delivering a nucleic acid into a cell using the compound. Here, the intracellular location may be the cytoplasm or the nucleus.

Hereinafter, preferred examples are presented to help understand the present invention. However, the following examples are provided only to help understand the present invention, and the present invention is not limited to the following examples.

### Synthesis Example

The Formula 1 (final compounds) of the present invention can be prepared by a reaction as in the following reaction scheme 1, but is not limited thereto.

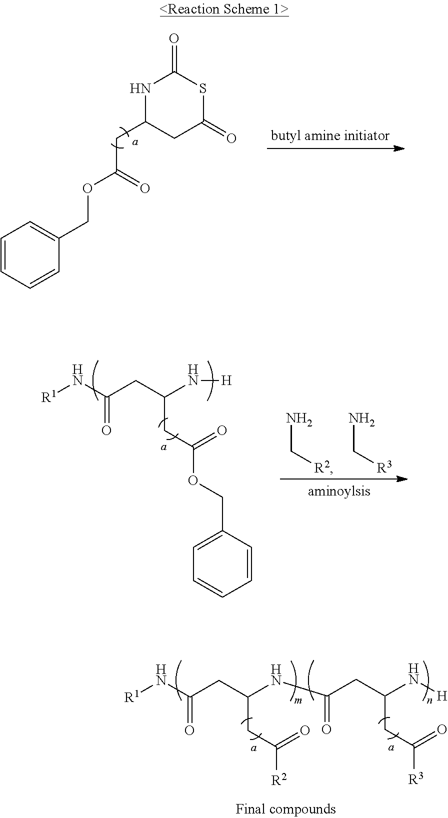

### Example 1. Synthesis of Poly(Beta-Amino Acids) Transformants

### 1-1. Synthesis of Benzyl-β³-LCHG Monomer

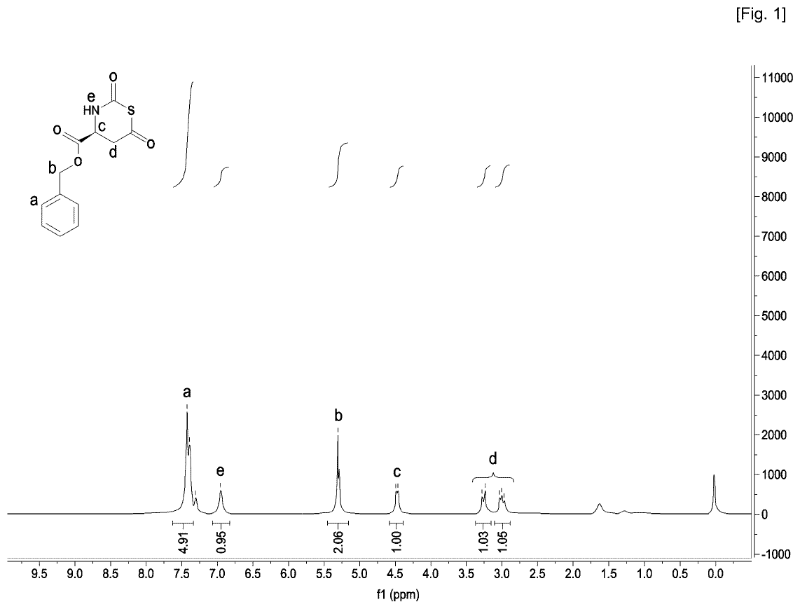

Benzyl-β³-LCHG monomer was synthesized using the same method based on a previous study (M. Zhou Angew. Chem. Int. Ed. 2020, 59, 7240-7244). The synthesis of the completed monomer was confirmed using ¹H-NMR (Avance III, 400 MHz, Bruker, Billerica, MA, USA) (FIG. 1).

### 1-2. Synthesis of Benzyl-β³-LCHA Monomer

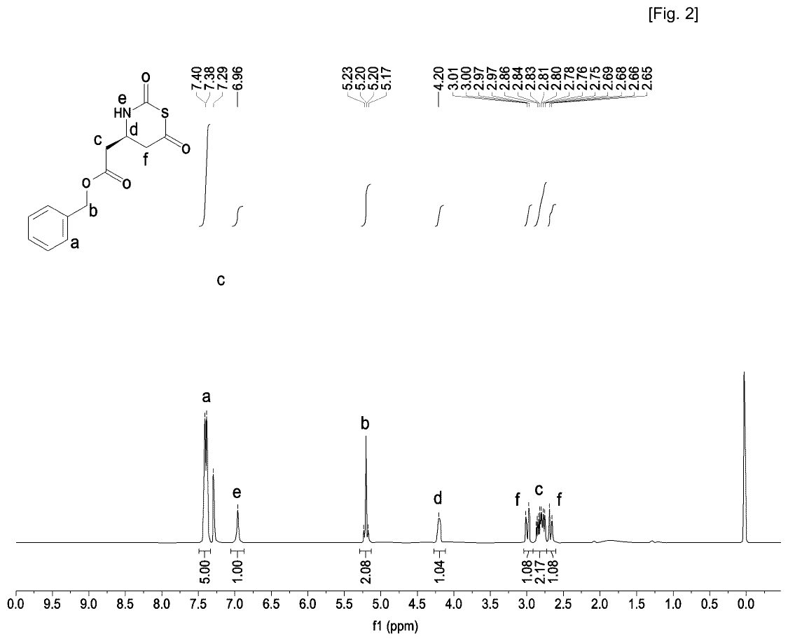

Benzyl-β³-LCHA monomer was synthesized using the same method based on a previous study (M. Zhou Angew. Chem. Int. Ed. 2020, 59, 7240-7244). The synthesis of the completed monomer was confirmed using ¹H-NMR (Avance III, 400 MHz, Bruker, Billerica, MA, USA) (FIG. 2).

1-3. Monomer (a=2) Synthesis

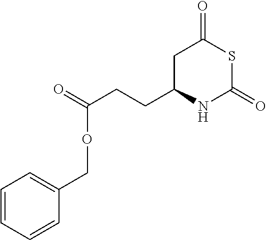 [SMILES: O=C(CC[C@H]1CC(=O)SC(=O)N1)OCc1ccccc1]

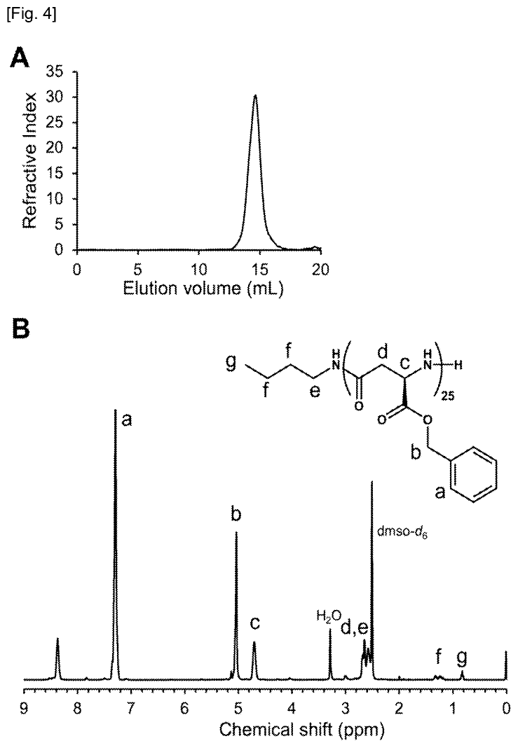

The monomer (a=2) represented by Formula was synthesized based on a previous study (M. Zhou Angew. Chem. Int. Ed. 2020, 59, 7240-7244). The synthesis of the completed monomer was confirmed using ¹H-NMR (Avance III, 400 MHz, Bruker, Billerica, MA, USA) (FIG. 4).

### 1-4. Synthesis of Poly(Benzyl-β³-LCHG) Precursor Polymer

The precursor polymer was synthesized as follows based on a previous study (M. Zhou Angew. Chem. Int. Ed. 2020, 59, 7240-7244).

Benzyl-β³-LCHG monomer (100 mg) was dissolved in DCM (2 mL), and acetic acid (3.45 μL) was added (D. Siefker ACS Macro Lett. 2018, 7, 1272-1277). The initiator n-butylamines (1.1 mg) dissolved in DCM was added to the monomer solution and stirred at 35° C. for 72 hours. The solution after the reaction was precipitated in hexane/ethyl acetate (6:4 v/v), filtered, and dried under reduced pressure.

The synthesized butyl-poly(benzyl-β³-LCHG) precursor polymer was analyzed using a gel permeation chromatography (HLC-8420, Tosoh corporation) equipped with 2 TSK columns (G3000HHR and G4000HHR) in DMF solution containing 10 mM lithium chloride, and the molecular weight distribution (Mw/Mn) was calculated. Additionally, the degree of polymerization (DP) was analyzed by ¹H-NMR. As a result, the Mw/Mn and DP of the butyl-poly(benzyl-β³-LCHG) precursor polymer were confirmed to be 1.041 and 25 (FIG. 4).

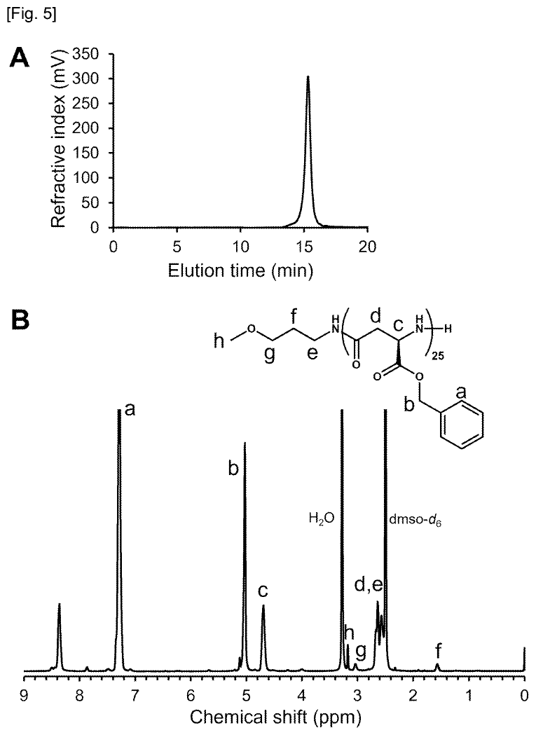

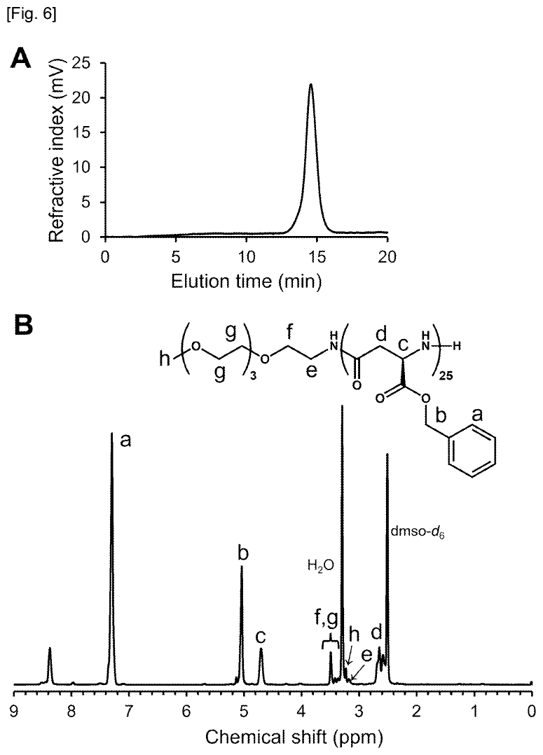

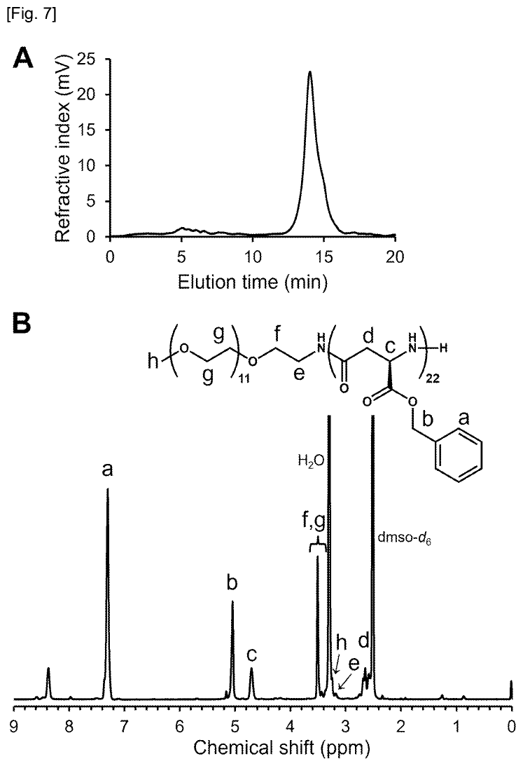

In the same manner as above, the initiator was changed to PEG₁-amine, PEG₄-amine, PEG₁₂-amine, and PEG₂₄-amine to prepare precursor polymers having different PEG lengths (FIGS. 5 to 7), and the Mw/Mn and DP of each poly(benzyl-β³-LCHG) precursor polymer are shown in Table 1.

TABLE 1

| Precursor polymer | Initiator | DP | M_{w}/Mₙ | Yield (%) |
|---|---|---|---|---|
| butyl-poly(benzyl-β³-LCHG) | butylamine | 25 | 1.041 | 94.4 |
| PEG₁-poly(benzyl-β³-LCHG) | PEG₁-amine | 25 | 1.031 | 85.6 |
| PEG₄-poly(benzyl-β³-LCHG) | PEG₄-amine | 25 | 1.032 | 86.0 |
| PEG₁₂-poly(benzyl-β³-LCHG) | PEG₁₂- | 22 | 1.041 | 90.0 |
| | amine | | | |
| PEG₂₄-poly(benzyl-β³-LCHG) | PEG₂₄- | 26 | 1.029 | 65.4 |
| | amine | | | |

### 1-5. Synthesis of Butyl-Poly(Benzyl-β³-LCHA) Precursor Polymer

Butyl-poly(benzyl-β³-LCHA) precursor polymer was synthesized using the same method as butyl-poly(benzyl-β³-LCHG) precursor polymer.

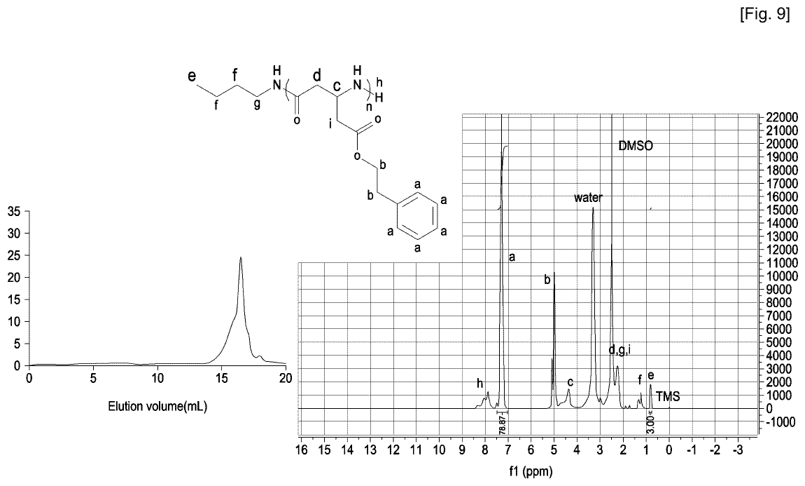

Benzyl-β³-LCHA monomer (100 mg) was dissolved in DCM (2 mL), and acetic acid (3.28 μL) was added. The initiator n-butylamines (1.047 mg) dissolved in DCM was added to the monomer solution and stirred at 35° C. for 72 hours. The solution after the reaction was precipitated in hexane/ethyl acetate (6:4 v/v), filtered, and dried under reduced pressure. The Mw/Mn and DP of the butyl-poly(benzyl-β³-LCHA) precursor polymer were confirmed to be 1.030 and 16 (FIG. 9).

### 1-6. Synthesis of Poly(Benzyl-β³-LCHG) Derivatives

The synthesized precursor polymer was converted into various poly(beta amino acids) derivatives through aminolysis reaction with compounds having various pKₐ and hydrophobicity.

### Synthesis Example of Butyl-PGly(DET/CHE)

The butyl-poly(benzyl-β³-LCHG) precursor polymer (20 mg) was dissolved in N-Methyl-2-pyrrolidone (NMP, 1 mL) at 35° C. and then cooled to 4° C. A solution of diethylenetriamine (DET, 0.26 mL) and cyclohexylethylamine (CHE, 0.93 mL) containing NMP (1 mL) was maintained at 10° C. and the precursor polymer solution was slowly added under argon gas. The reaction solution was reacted at 10° C. for 24 hours. After the reaction was completed, the reaction solution was precipitated in ethyl ether to obtain a powder. After dissolving this powder in cold 0.01 M HCl, dialysis was performed using 0.01 M HCl for 2 days at 4° C. Finally, the final product in the form of powder was obtained by freezing and drying the solution after dialysis with only water for 1-2 hours. The amount of DET and CHE introduced into the manufactured polymers was calculated using ¹H-NMR.

The synthesis reaction conditions for each transformant with different PEG lengths are presented in Table 2, and the introduction amounts of the manufactured polymers are shown in Table 3.

TABLE 2

| | Precursor | | | | |
|---|---|---|---|---|---|
| | polymer | Molar ratio (pKa | pKa | Hydrophobic | |
| Precursor | reaction | molecule:hydrophobic | molecule | molecules | Yield |
| polymer | amount (mg) | molecule) | and dosage | and dosage | (mg) |
| butyl- | 20 | 25:65 | DET, 263.5 | CHE, 927.4 | 14 |
| poly(benzyl-β³- | | | μl | μl | |
| LCHG) | | | | | |
| PEG₁- | 20 | 25:65 | DET, 263.5 | CHE, 927.4 | 17 |
| poly(benzyl-β³- | | | μl | μl | |
| LCHG) | | | | | |
| PEG₄- | 20 | 25:65 | DET, 263.5 | CHE, 927.4 | 17 |
| poly(benzyl-β³- | | | μl | μl | |
| LCHG) | | | | | |
| PEG₁₂- | 18.8 | 25:70 | DET, 221.4 | CHE, 839.2 | 13 |
| poly(benzyl-β³- | | | μl | μl | |
| LCHG) | | | | | |
| PEG₂₄- | 20.2 | 25:70 | DET, 223.7 | CHE, 847.94 | 7 |
| poly(benzyl-β³- | | | μl | μl | |
| LCHG) | | | | | |
| butyl- | 20 | 25:80 | DET, 263.5 | ADO, 1.35 g | 15 |
| poly(benzyl-β³- | | | μl | | |
| LCHG) | | | | | |
| PEG₁- | 20 | 25:65 | DET, 263.5 | ADO, 1.1 g | 15 |
| poly(benzyl-β³- | | | μl | | |
| LCHG) | | | | | |
| PEG₄- | 12 | 25:80 | DET, 158.1 | ADO, 0.81 g | 10 |
| poly(benzyl-β³- | | | μl | | |
| LCHG) | | | | | |
| butyl- | 20 | 100:160 | AEP, 1281 | CHE, 2283 | 9 |
| poly(benzyl-β³- | | | μl | μl | |
| LCHG) | | | | | |
| butyl- | 20 | 25:65 | HED, 493 | CHE, 927.4 | 8 |
| poly(benzyl-β³- | | | μl | μl | |
| LCHG) | | | | | |
| butyl- | 20 | 25:65 | MDE, 314.1 | CHE, 927.4 | 17 |
| poly(benzyl-β³- | | | μl | μl | |
| LCHG) | | | | | |

TABLE 3

| | Ethylene glycol | Introduction amount | |
|---|---|---|---|
| Polymer | repeating number | DET moiety | CHE moiety |
| Butyl-PGly(DET/CHE) | 0 | 12 | 13 |
| PEG₁-PGly(DET/CHE) | 1 | 10 | 16 |
| PEG₄-PGly(DET/CHE) | 4 | 13 | 12 |
| PEG₁₂-PGly(DET/CHE) | 12 | 11 | 11 |
| PEG₂₄-PGly(DET/CHE) | 24 | 12 | 14 |

Poly(beta-amino acids) derivatives with additional pKₐ and hydrophobicity were synthesized in the same manner as the above synthetic method, and the chemical transformations of each pKₐ and hydrophobic group are shown in Table 4.

TABLE 4

| | | pKₐ chemical group | | hydrophobic chemical group | |
|---|---|---|---|---|---|
| | | | Introduction | | Introduction |
| | Polymer | chemical group | amount | chemical group | amount |
| Hydrophobic | Butyl- | diethylenetriamine | 13 | 10-amino-1- | 12 |
| control | PGly(DET/ | (DET) | | decanol (ADO) | |
| | ADO) | | | | |
| | PEG₁- | diethylenetriamine | 14 | 10-amino-1- | 11 |
| | PGly(DET/ | (DET) | | decanol (ADO) | |
| | ADO) | | | | |
| | PEG₄- | diethylenetriamine | 14 | 10-amino-1- | 11 |
| | PGly(DET/ | (DET) | | decanol (ADO) | |
| | ADO) | | | | |
| pKa | Butyl- | 2- | 8 | cyclohexylethylamine | 17 |
| regulation | PGly(AEP/ | aminoethylpiperazine | | (CHE) | |
| | CHE) | (AEP) | | | |
| | Butyl- | (2- | 10 | cyclohexylethylamine | 15 |
| | PGly(HED/ | hydroxyethyl)ethylene- | | (CHE) | |
| | CHE) | diamine (HED) | | | |
| | Butyl- | 2,2′-diamino-N- | 13 | cyclohexylethylamine | 12 |
| | PGly(MDE/ | methyldiethylamine | | (CHE) | |
| | CHE) | (MDE) | | | |

### Example 2. Measurement of Binding of Poly(Benzyl-β³-LCHG) Derivatives to mRNA

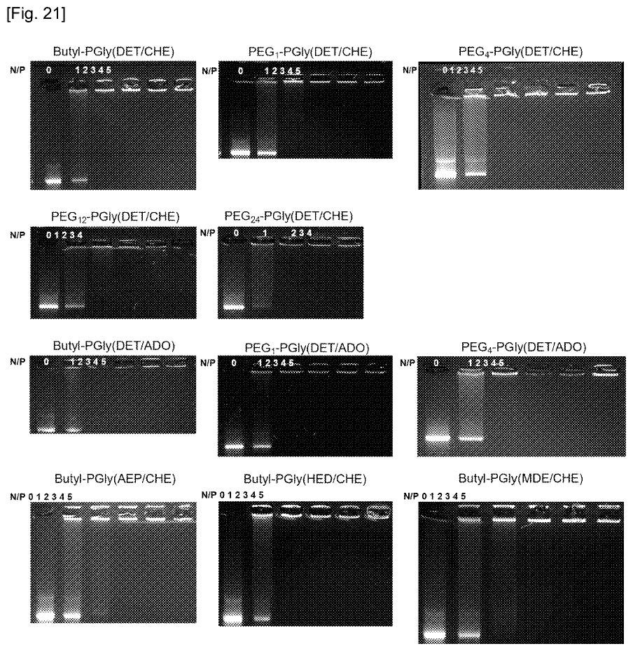

Each polymer was dissolved at 2 mg/mL in 10 mM Hepes (pH 7.3) buffer and then diluted again in 10 mM Hepes (pH 7.3) buffer so that the molar concentration of the protonable amino group was 0.624 mM. A solution prepared with luciferase mRNA (L-7202, TriLink Biotechnologies, San Diego, CA, USA) (100 ng/μL) was mixed so that the molar concentration of protonable amino group/molar concentration of phosphate group (N/P ratio)=1 to 5, and incubated at room temperature for approximately 1 hour. After preparing a 1.0% agarose gel, polymer nanoparticles (10 μL) and glycerol solution (50% v/v) (2 μL) were mixed and electrophoresis (135 V, 20 min) was performed. The agarose gel after electrophoresis was observed using a gel imager (FIG. 21). As a result, it was confirmed that the association between the polymer derivative of the present invention and mRNA was excellent.

### Example 3. Measurement of the Size and Surface Charge of Polymer Nanoparticles of Poly(Benzyl-β³-LCHG) Derivatives and mRNA

After manufacturing polymer nanoparticles containing luciferase mRNA at N/P=5 by the method, the size (D_{H}), polydispersity index (PDI), and surface charge (ξ-potential) of the nanoparticles were measured using a Zetasizer Pro (RED) (Malvern Instruments, Worcestershire, UK), and the measured values are shown in Table 5.

TABLE 5

| | | | | | ξ- |
|---|---|---|---|---|---|
| | | | | | poten- |
| | | N/P | D_{H} | | tial |
| | Polymer | ratio | (nm) | PDI | (mV) |
| — | Butyl- | 5 | 119 ± 7 | 0.12 ± 0.01 | 22.16 |
| | PGly(DET/CHE) | | | | |
| PEG | PEG₁- | 5 | 106 ± 5 | 0.16 ± 0.03 | 28.65 |
| Length | PGly(DET/CHE) | | | | |
| adjust- | PEG₄- | 5 | 120 ± 12 | 0.14 ± 0.03 | 27 |
| ment | PGly(DET/CHE) | | | | |
| | PEG₁₂- | 5 | 202 ± 19 | 0.22 ± 0.01 | 16.95 |
| | PGly(DET/CHE) | | | | |
| | PEG₂₄- | 5 | 130 ± 12 | 0.17 ± 0.04 | 26.13 |
| | PGly(DET/CHE) | | | | |
| Hydropho- | Butyl- | 5 | 171 ± 10 | 0.10 ± 0.01 | 29.21 |
| bicity | PGly(DET/ADO) | | | | |
| Control | PEG₁- | 5 | 187 ± 6 | 0.09 ± 0.01 | 25.4 |
| | PGly(DET/ADO) | | | | |
| | PEG₄- | 5 | 316 ± 19 | 0.20 ± 0.01 | 20.54 |
| | PGly(DET/ADO) | | | | |
| pKa | Butyl- | 5 | 191 ± 25 | 0.26 ± 0.09 | 36.74 |
| Control | PGly(AEP/CHE) | | | | |
| | Butyl- | 5 | 145 ± 4 | 0.13 ± 0.03 | 44.08 |
| | PGly(HED/CHE) | | | | |
| | Butyl- | 5 | 176 ± 17 | 0.21 ± 0.04 | 43.83 |
| | PGly(MDE/CHE) | | | | |

<Hydrodynamic diameter (D_{H}), PDI, surface charge (N/P = 5) of polymer nanoparticles>

Also, similarly, polymer nanoparticles containing luciferase mRNA were prepared at N/P=7, and the size, polydispersity index (PDI), and surface charge (ξ-potential) of the nanoparticles were measured using Zetasizer Pro (RFD), and the measured values are shown in Table 6.

TABLE 6

| | | | | | ξ- |
|---|---|---|---|---|---|
| | | | | | poten- |
| | | N/P | D_{H} | | tial |
| | Polymer | ratio | (nm) | PDI | (mV) |
| — | Butyl- | 7 | 87 ± 4 | 0.13 ± 0.02 | 28.7 |
| | PGly(DET/CHE) | | | | |
| PEG | PEG₁- | 7 | 129 ± 3 | 0.12 ± 0.04 | 36.4 |
| Length | PGly(DET/CHE) | | | | |
| adjustment | PEG₄- | 7 | 77 ± 9 | 0.13 ± 0.01 | 27.1 |
| | PGly(DET/CHE) | | | | |
| | PEG₁₂- | 7 | 89 ± 6 | 0.16 ± 0.02 | 18.7 |
| | PGly(DET/CHE) | | | | |
| | PEG₂₄- | 7 | 87 ± 19 | 0.15 ± 0.05 | 21.7 |
| | PGly(DET/CHE) | | | | |

<Hydrodynamic diameter (D_{H}), PDI, surface charge (N/P = 7) of polymer nanoparticles>

### Example 4. Transmission Electron Microscope (TEM) Measurements

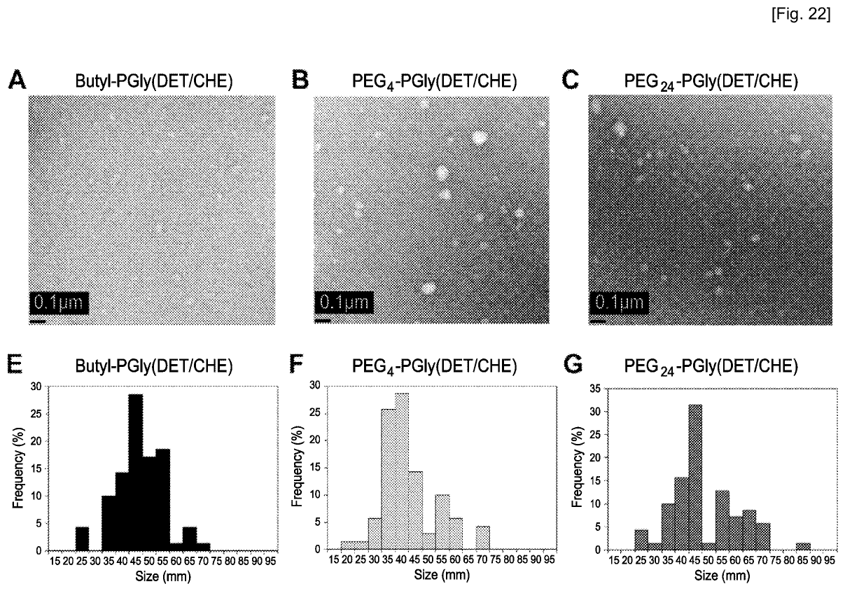

Polymer nanoparticles (N/P=5) were prepared using luciferase mRNA and polymers of different PEG lengths (butyl-PGly(DET/CHE), PEG₄-PGly(DET/CHE), PEG₂₄-PGly(DET/CHE)). The manufactured polymer nanoparticles were placed on 400 mesh copper grids (Electron Microscopy Sciences, Pennsylvania, USA), stained with UranyLess EM Stain solution (Electron Microscopy Sciences), and measured using a field emission transmission electron microscope (FE-TEM, JEM2100F, JEOL Ltd., Tokyo, Japan) (FIGS. 22A to C). The measured images were used to measure the average diameter of nanoparticles using ImageJ software (FIGS. 22D to F). The average diameters of butyl-PGly(DET/CHE), PEG₄-PGly(DET/CHE), and PEG₂₄-PGly(DET/CHE) polymeric nanoparticles were measured as 50±9, 45±10, and 52±13 nm, respectively (n=70).

### Example 5. Measurement of Luciferase mRNA Expression Level of Polymer Nanoparticles in C2C12 Cells

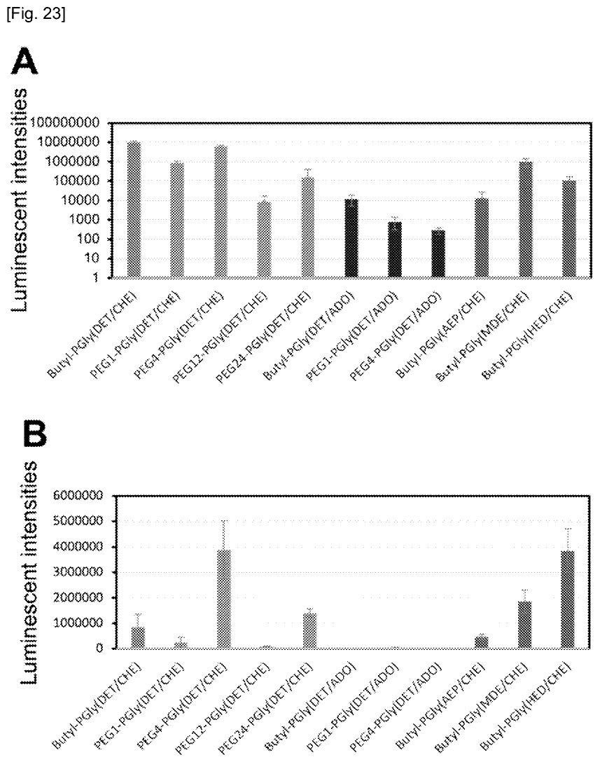

Polymeric nanoparticles containing luciferase mRNA were transfected into mouse myoblast cells, C2C12 cells, and the luciferase mRNA delivery ability was measured. C2C12 cells were prepared using DMEM medium supplemented with 10% FBS. C2C12 cells were seeded at 8,000 cells/well in a 96-well plate and cultured in a CO₂ chamber for one day. Polymer nanoparticles (N/P=5) were transfected into C2C12 cells at 50 ng mRNA/well. The cells were incubated for 24 h and lysed using a lysis buffer. The cell lysates were used to measure a luminescence intensity using Luciferase Assay System (Promega, Madison, WI, USA) and luminescence microplate reader (Mithras LB940, Berthold technologies, Bad Wildbad, Germany). Transfection of polymer nanoparticles was measured in 2 conditions: with 10% FBS (FIG. 23A) and without 10% FBS (FIG. 23B) to examine the PEG effect in more detail. Butyl-, PEG1-, and PEG₄-PGly(DET/CHE) polymers showed high luciferase mRNA expression levels.

### Example 6. Analysis of Cellular Uptake and Serum Stability of Polymer Nanoparticles in C2C12 Cells

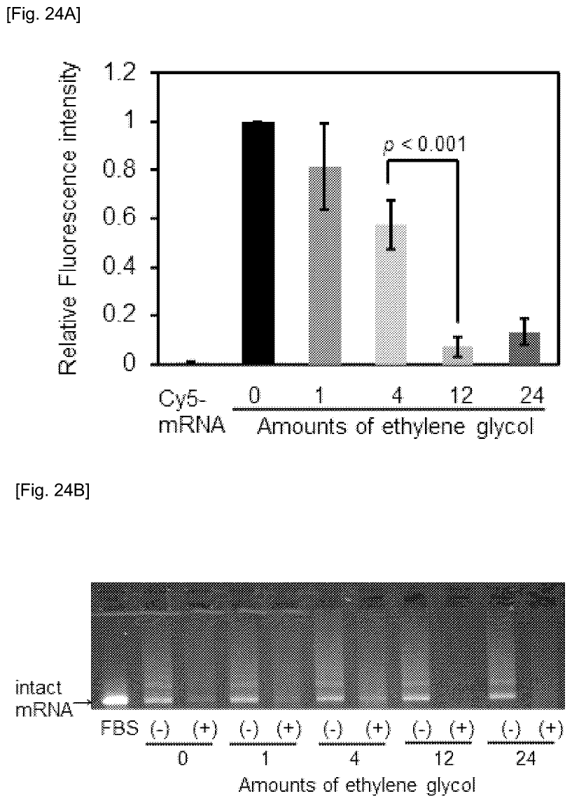

To further investigate the PEG length effect of PEGₙ-PGly(DET/CHE) polymers, the cellular uptake of polymer nanoparticles into C2C12 cells was analyzed. Using the Label IT Cy5 labeling kit (Mirus Bio Corporation, Madison, WI, USA), Cy5-labeled luciferase mRNA was prepared by reacting according to the protocol provided in the kit (Cy5-mRNA). Polymer nanoparticles containing Cy5-mRNA (N/P=5, 125 ng mRNA/well) were transfected into C2C12 cells seeded in 48-well plates (50,000 cells/well). After 4 hours of incubation, cells were detached using trypsin-EDTA, and the cellular uptake of polymer nanoparticles was compared by comparing the amount of fluorescence expression using flow cytometry (Attune CytPix, ThermoFisher Scientific) (FIG. 24A). Through this, it was confirmed that a significant amount of Cy5-mRNA was taken up by C2C12 cells for polymers with PEG lengths of 0-4. In contrast, it was confirmed that in the case of polymers with PEG lengths of 12 and 24, almost no cellular uptake occurred.

To examine in more detail the effect of PEG length on cellular uptake of polymer nanoparticles, the particle stability of the same polymer nanoparticles against serum albumin was measured using fetal bovine serum (FBS). Polymer nanoparticles (N/P=5, 100 ng mRNA, 10 uL) were mixed with PBS solution containing 10% FBS (PBS/FBS, 90 uL) and incubated at 37° C. for 1 hour. After 1 hour, mRNA was purified using the RNeasy Mini kit (Qiagen). After performing agarose gel electrophoresis (1 wt % agarose gel, 0.5×TAE buffer, 135 V, 15 min), the bands were observed with a gel imager (WSE-5300 Printgraph CMOS 1) (FIG. 24B). For butyl-, PEG₁-, PEG₄-PGly(DET/CHE) polymer nanoparticles, intact mRNA bands are observed in the solution in FBS. This result indicates that the polymer nanoparticles are stable in FBS. In contrast, in the case of PEG₁₂-, PEG₂₄-PGly(DET/CHE) polymer nanoparticles, the intact mRNA band was not observed. This means that both polymer nanoparticles degrade within 1 hour in PBS/FBS, mimicking cell culture. Through these results, we confirmed that in the case of PGly(DET/CHE) polymers, when the PEG length is 12 or longer, the nanoparticles are unstable in the cell culture environment, quickly degrade, and cannot deliver mRNA into cells.

### Example 7. Viability Assay for C2C12 Cells

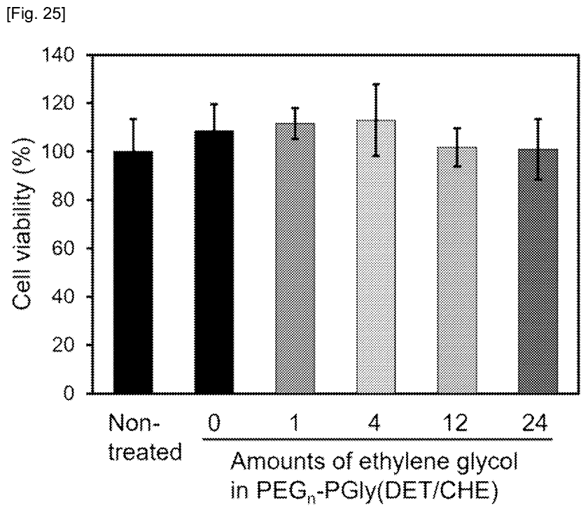

C2C12 cells were seeded in 96-well plates (8,000 cells/well) and transfected with PEGₙ-PGly(DET/CHE) polymer nanoparticles containing luciferase mRNA (N/P=5, 50 ng/well) the next day. After 24 hours of incubation, 10 μL of CCK-8 solution was added to each well, and the absorbance at 450 nm was measured 1.5 hours later using a microplate reader (Spark, Tecan Group Ltd., Mannedorf, Switzerland) (FIG. 25). In this experimental environment, PEGₙ-PGly(DET/CHE) polymer nanoparticles did not show significant cytotoxicity against C2C12.

### Example 8. Confirmation of the Gene Editing Effect of PEGₙ-PGly(DET/CHE) Polymer in HEK293-loxP-GFP-RFP Cells

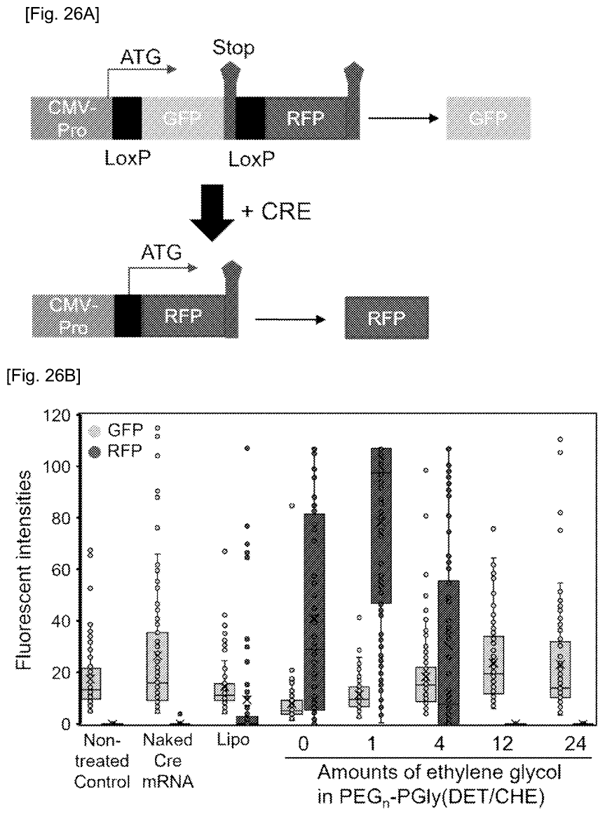

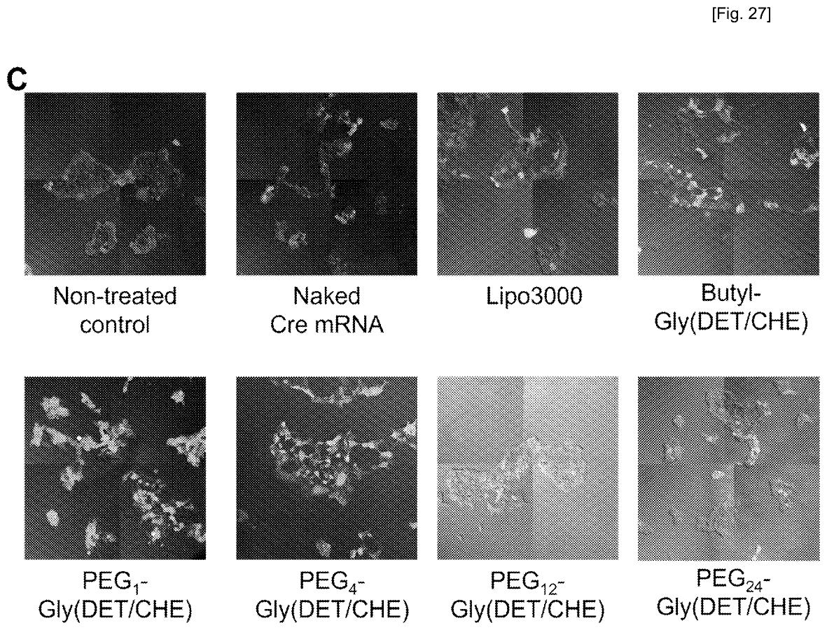

HEK293-loxP-GFP-RFP cells (GenTarget Inc, SC018-Neo, San Diego, CA, USA) have a structure in which the loxP-GFP-stop-loxP-RFP cassette is located behind the CMV-protomer and normally exhibit strong green fluorescence, but when loxP is cut by CRE recombinase, it exhibits red fluorescence (FIG. 26A). This was used to measure how well polymer nanoparticles deliver Cre mRNA (L-7211, TriLink Biotechnologies) into cells to achieve gene editing efficiency. Cells were seeded in 96-well optical bottom plates (8,000 cells/well) and transfected with PEGₙ-PGly(DET/CHE) polymer nanoparticles (N/P=5, 100 ng/uL). After 48 hours of incubation, green (GFP) and red (RFP) fluorescence was measured using a confocal laser scanning microscope (CLSM) (ZEISS LSM 980, Carl Zeiss, Oberkochen, Germany) (FIG. 27), and each fluorescence intensity was analyzed and quantified using Image J software (FIG. 26B). The high Cre mRNA delivery ability of the 3 polymers was confirmed by high RFP fluorescence in cells treated with butyl-, PEG₁-, and PEG₄-PGly(DET/CHE) polymer nanoparticles. In particular, it was confirmed that PEG₁-PGly(DET/CHE) polymer nanoparticles showed the highest gene editing effect.

In summary, in the present invention, a novel poly(beta-amino acids) derivatives, which has never been used for RNA drug delivery before, was synthesized having with various pKₐ, hydrophobicity, and PEG length. Furthermore, the synthesized polymer was successfully associated with mRNA and nanoparticles with a size of 100-300 nm were prepared. Moreover, from the above results, it was confirmed that the nanoparticles of the present invention successfully delivered luciferase mRNA to C2C12 cells in vitro, and the possibility of RNA drug delivery of poly(beta-amino acids) derivatives was confirmed by comparatively analyzing the gene editing efficiency by delivering Cre mRNA to HEK293-loxP-GFP-RFP cells using the nanoparticles of the present invention.

The above description is merely an example of the present invention, and those skilled in the art will appreciate that various modifications may be made without departing from the essential characteristics of the present invention. Accordingly, the embodiments disclosed in this specification are intended to illustrate rather than limit the present invention, and the spirit and scope of the present invention are not limited by these embodiments. The scope of protection of the present invention should be interpreted by the claims below, and all technologies within a scope equivalent thereto should be interpreted as being included in the scope of the rights of the present invention.

### INDUSTRIAL APPLICABILITY

According to the present invention, an amphiphilic polymer compound having properties such as an easy manufacturing process, high binding capacity with therapeutic nucleic acids, excellent stability in blood, and delivery ability can be manufactured, and thus has industrial applicability.

1. A compound represented by Formula 1: 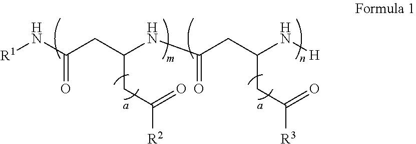
wherein:
R¹ is an C₁-C₁₀ alkyl group; —(CH₂)₃—O—CH₃; or —(CH₂)ₓ—[(CH₂)₂—O]_{y}—CH₃;
x is 0 or 1, y is an integer from 1 to 45, provided that where y is 1, x is 1, and where y is 2 or more, x is 0,
m and n are each independently an integer from 1 to 100, provided that m+n≥10,
a is an integer from 0 to 2, and
R² and R³ are each independently represented by Formula 2, 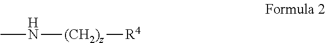
wherein:
R⁴ is selected from the group consisting of hydrogen; an amino group; a hydroxyl group; a C₁-C₂₀ alkoxy group; a C₃-C₂₀ aliphatic ring group; a C₆-C₂₀ aryl group; a C₁-C₂₀ heteroaryl group; a C₁-C₂₀ heterocyclic group; —NH—C₁˜C₂₀ alkenyl group-NH₂; —NH—C₁-C₂₀ hydroxyalkyl group; —N(C₁-C₂₀ alkyl group)(C₁-C₂₀ alkyl group); —N(C₁-C₂₀ alkyl group)(C₁-C₂₀ alkenyl group-NH₂); —(OH)(C₁-C₂₀ alkyl group); and —(C₁-C₂₀ hydroxyalkyl group)(C₁-C₂₀ hydroxyalkyl group);
z is an integer from 0 to 45.

2. The compound according to claim 1, wherein R¹ is represented by any one of Formulas 1-1 to 1-3: 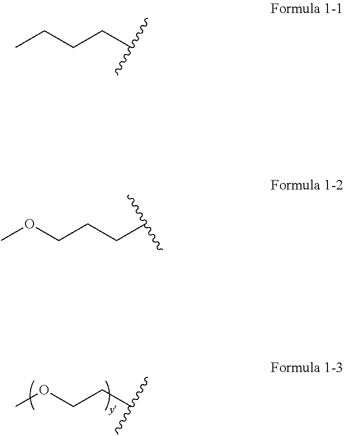
wherein:
y′ is an integer from 2 to 45, and [symbol]
indicates the position to be bonded.

3. The compound according to claim 1, wherein R² or R³ is represented by any one of Formulas 2-1 to 2-20: 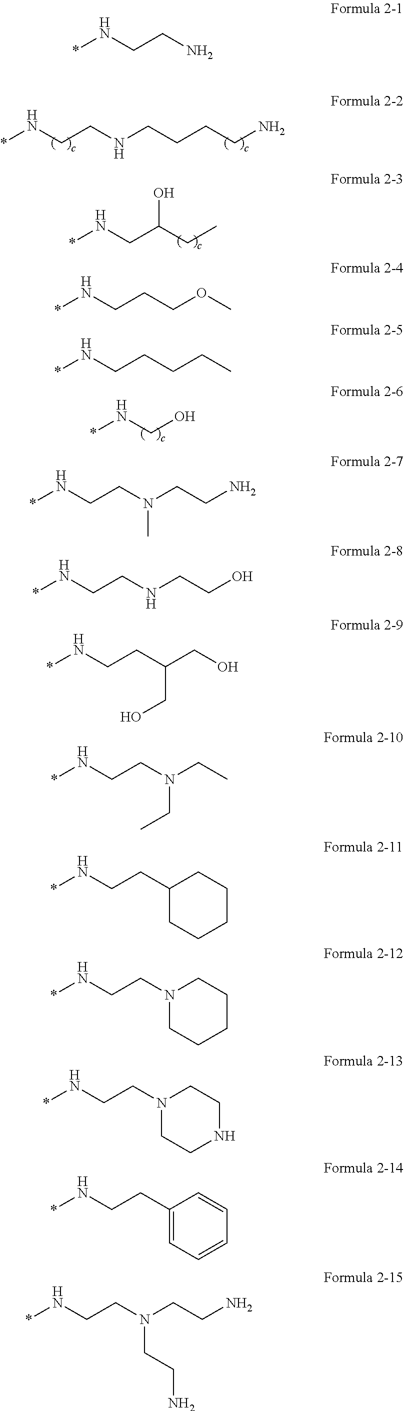 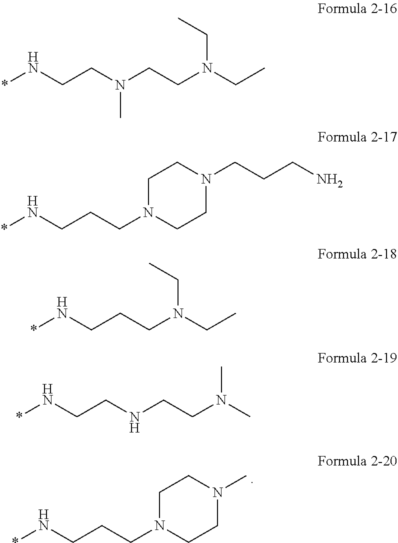
wherein:
c is an integer from 0 to 12.

4. A composition for nucleic acids delivery comprising a compound of claim 1 and a nucleic acid.

5. The composition for nucleic acids delivery according to claim 4, wherein the nucleic acid is selected from the group consisting of DNA, RNA, PNA, interfering RNA (siRNA), asymmetric interfering RNA (aiRNA), microRNA (miRNA), dicer-substrate RNA (dsRNA), small hairpin RNA (shRNA), messenger RNA (mRNA), transfer RNA (tRNA), ribosomal RNA (rRNA), antisense oligonucleotides (ASOs) and mixtures thereof.

6. The composition for nucleic acids delivery according to claim 4, further comprising a pharmaceutically acceptable carrier or a pharmaceutically acceptable salt.

7. A nanoparticle formed by combination of the compound of claim 1 with a nucleic acid.

8. The nanoparticle according to claim 7, wherein a molar ratio of protonable amino groups in the compound to phosphate groups in the nucleic acid (N/P ratio) is 1 to 30.

9. A vaccine composition comprising the nanoparticle according to claim 7.

10. A method for producing the compound represented by Formula 1 of claim 1, comprising
1) a step of synthesizing a beta-amino acid N-thiocarboxyanhydrides (beta-NTAs) monomer;
2) a step of synthesizing a polymer precursor by ring-opening polymerization of the beta-NTAs monomer using butylamine as an initiator;
3) a step of modifying a side chain of the polymer precursor through an aminolysis reaction.

11. A method of delivering a nucleic acid into a cell using the compound of claim 1.

12. The method of claim 11, wherein the intracellular location is the cytoplasm or the nucleus.
